(19) United States
(12) Patent Application Publication
(10) Pub. No.: US 2025/0169771 A1
(43) Pub. Date: May 29, 2025

(54) CONTROL DEVICE FOR VIBRATOR OF HAPTIC SENSATION PRESENTATION DEVICE, AND HAPTIC SENSATION PRESENTATION DEVICE

(71) Applicant: Murata Manufacturing Co., Ltd., Kyoto (JP)

(72) Inventor: Norio NAKAMURA, Kyoto (JP)

(21) Appl. No.: 19/042,148

(22) Filed: Jan. 31, 2025

Related U.S. Application Data
(63) Continuation of application No. PCT/JP2023/027176, filed on Jul. 25, 2023.

(30) Foreign Application Priority Data
Aug. 1, 2022 (JP) 2022-122720

(51) Int. Cl. A61B 5/00

(52) U.S. Cl. CPC A61B 5/7455; A61B 5/746; A61B 2505/09

(57) ABSTRACT

A control device includes a CPU used as an execution device and a storage device. The storage device stores multiple vibration modes for haptic sensations that are presented by a haptic sensation presentation device for rehabilitation. The CPU performs a preliminary drive process, a parameter acquisition process, and a mode selection process. In the preliminary drive process, the CPU drives the vibrator in the multiple vibration modes to present haptic sensations corresponding to the vibration modes. In the parameter acquisition process, the CPU obtains effect parameters for the respective vibration modes. The effect parameters indicate the effectiveness of the rehabilitation on the user achieved when the vibrator is driven in the respective vibration modes. In the mode selection process, the CPU selects a specific mode from the multiple vibration modes based on the effect parameters obtained in the respective vibration modes.

### CROSS REFERENCE TO RELATED APPLICATION

This is a continuation of International Application No. PCT/JP2023/027176 filed on Jul. 25, 2023 which claims priority from Japanese Patent Application No. 2022-122720 filed on Aug. 1, 2022. The contents of these applications are incorporated herein by reference in their entireties.

### BACKGROUND ART

### Technical Field

The present disclosure relates to a control device for a vibrator of a haptic sensation presentation device, and the haptic sensation presentation device.

A haptic sensation presentation device described in Patent Document 1 includes a housing, a vibrator, and a control device. The vibrator is disposed inside of the housing. When using the haptic sensation presentation device, the user holds the housing in hand. The control device controls the vibrator in a specific vibration mode. With this configuration, the haptic sensation presentation device presents various haptic sensations to the user touching the housing.

Patent Document 1: Japanese Unexamined Patent Application Publication No. 2005-190465

### BRIEF SUMMARY

As an example of a situation in which the haptic sensation presentation device as described in Patent Document 1 is used, the haptic sensation presentation device may be used as an assistive device in rehabilitation for improving a motor function of a user. However, it is unclear what haptic sensations need to be presented to the user in such a situation to enhance the effect of improving the motor function.

To solve the above problem, an aspect of the present disclosure provides a control device for a vibrator of a haptic sensation presentation device. The control device includes a storage device and an execution device. The storage device stores multiple vibration modes of the vibrator that are used to present haptic sensations output by the haptic sensation presentation device for rehabilitation. The execution device performs a preliminary drive process of driving the vibrator in the multiple vibration modes to present the haptic sensations in at least two vibration modes among the multiple vibration modes, a parameter acquisition process of obtaining effect parameters for the respective vibration modes, the effect parameters indicating the effectiveness of the rehabilitation on a user achieved when the vibrator is driven in the respective vibration modes in the preliminary drive process, and a mode selection process of selecting a specific mode, which is a specific vibration mode, from among the multiple vibration modes based on the effect parameters obtained in the respective vibration modes in the parameter acquisition process.

To solve the above problem, an aspect of the present disclosure provides a haptic sensation presentation device including a vibrator and a control device that includes a storage device and an execution device and controls the vibrator. The storage device stores multiple vibration modes of the vibrator that are used to present haptic sensations output for rehabilitation. The execution device performs a preliminary drive process of driving the vibrator in the multiple vibration modes to present the haptic sensations in at least two vibration modes among the multiple vibration modes, a parameter acquisition process of obtaining effect parameters for the respective vibration modes, the effect parameters indicating the effectiveness of the rehabilitation on the user achieved when the vibrator is driven in the respective vibration modes in the preliminary drive process, and a mode selection process of selecting a specific mode, which is a specific vibration mode, from among the multiple vibration modes based on the effect parameters obtained in the respective vibration modes in the parameter acquisition process.

According to each of the above configurations, the specific mode is selected based on the effectiveness of rehabilitation performed on the user in the preliminary drive process. This enables the control device to select an appropriate specific mode from among the multiple vibration modes based on the effectiveness of the rehabilitation on the user.

The present disclosure makes it possible to select an appropriate specific mode based on the effectiveness of rehabilitation of a user.

### BRIEF DESCRIPTION OF THE DRAWINGS

FIG. 1 is a schematic diagram illustrating a haptic sensation presentation system.

FIG. 2 is a flowchart illustrating a series of processes performed by a control program.

### DETAILED DESCRIPTION

### Embodiment

A control device for a haptic sensation presentation device according to an embodiment is described with reference to the drawings. First, a haptic sensation presentation system including a haptic sensation presentation device is described.

### Haptic Sensation Presentation System

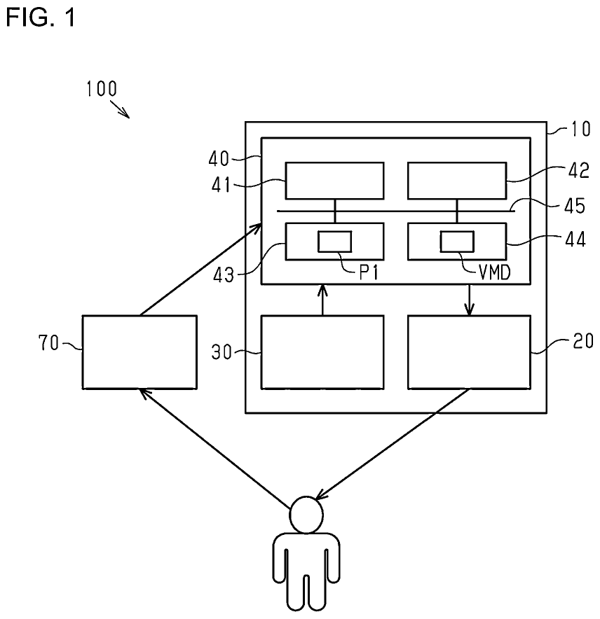

As illustrated in FIG. 1, a haptic sensation presentation system 100 includes a haptic sensation presentation device 10 and a measurement device 70. The haptic sensation presentation device 10 includes a vibrator 20, an input/output device 30, and a control device 40.

Although not illustrated in FIG. 1, the vibrator 20 is housed in a housing of the haptic sensation presentation device 10. Also, the vibrator 20 includes voice coil motors, weights for the respective voice coil motors, and a cubic case for housing these components. Each weight is caused to vibrate by a force generated when an electric current flows through the coil of the corresponding voice coil motor. When the weights vibrate, the case vibrates due to the vibration of the weights. Accordingly, the vibrator 20 is caused to vibrate in directions along axes orthogonal to the surfaces of the case by controlling electric currents flowing through the coils of the voice coil motors. More specifically, the vibrator 20 may be implemented by, for example, a vibrator described in Japanese Unexamined Patent Application Publication No. 2005-190465.

The input/output device 30 is used by the user to input information indicating a target part TP on which rehabilitation is to be performed. The input/output device 30 is implemented by, for example, a touch display. Therefore, the input/output device 30 can also provide information to the user via images. The input/output device 30 is operated by the user. The user operates the input/output device 30 to input information indicating the target part TP.

The control device 40 controls the vibrator 20. The control device 40 controls a pattern of vibration of the vibrator 20 according to a specific vibration pattern corresponding to a haptic sensation to be presented. Here, a vibration pattern indicates a pattern of vibrations represented by, for example, a non-linear waveform. With this configuration, the haptic sensation presentation device 10 presents a tactile sensation or a force sensation to the user. Here, haptic sensations include haptic illusions. Haptic illusions include a tactile illusion and a force illusion. A tactile illusion is an illusion in which the brain of the user feels as if the user is touching an uneven object when the vibrations of the vibrator 20 are presented to the user. A force illusion is an illusion in which the brain of the user feels as if a force is being applied to the user when the vibrations of the vibrator 20 are presented to the user.

The control device 40 includes a CPU 41 as an execution device, a peripheral circuit 42, a ROM 43, a storage device 44, and a bus 45. The bus 45 connects the CPU 41, the peripheral circuit 42, the ROM 43, and the storage device 44 to each other so that they can communicate with each other. The peripheral circuit 42 includes a circuit for generating a clock signal that regulates internal operations, a power supply circuit, and a reset circuit. The ROM 43 stores various programs that are executed by the CPU 41 to perform various control processes. In particular, the ROM 43 stores a control program P1 used to perform control processes of the vibrator 20 including a preliminary drive process and a mode selection process described later. The CPU 41 controls the vibrator 20 by executing various programs stored in the ROM 43.

The storage device 44 stores vibration mode data VMD indicating multiple vibration modes VM for rehabilitation. Each vibration mode VM is a mode of the vibrator 20 for presenting a haptic sensation that is output by the haptic sensation presentation device 10 for rehabilitation.

The storage device 44 also stores data indicating vibration patterns of the vibrator 20 for implementing the vibration modes VM. Haptic sensations presented by the haptic sensation presentation device 10 are determined by the data stored in the storage device 44.

The measurement device 70 measures activation parameters indicating active states of the user to which haptic sensations are presented by the haptic sensation presentation device 10. For example, the measurement device 70 measures the active states of the brain of the user. Specifically, the measurement device 70 is a brain measuring instrument using near-infrared spectroscopy.

The measurement device 70 measures activation parameters of respective regions of the brain. For example, the measurement device 70 measures the blood flow rate in each region of the brain as an activation parameter. A higher blood flow rate indicates a higher activation level of the brain. The measurement device 70 measures the activation parameters of regions of the primary motor cortex of the brain corresponding to body parts. The measurement device 70 transmits the activation parameters of regions of the brain to the control device 40.

### Control of Vibrator

The CPU 41 executes the control program P1 stored in the ROM 43 to perform a series of processes for controlling the vibrator 20. With this configuration, the CPU 41 performs an acquisition process, a preliminary drive process, a parameter acquisition process, a mode selection process, and a main drive process described later.

When the power supply of the haptic sensation presentation device 10 is turned on, the CPU 41 executes the control program P1 stored in the ROM 43. For example, the power supply of the haptic sensation presentation device 10 is turned on when the input/output device 30 is operated while the power supply of the haptic sensation presentation device 10 is in the off state. The control program P1 causes the CPU 41 to perform the acquisition process, the preliminary drive process, the parameter acquisition process, the mode selection process, and the main drive process.

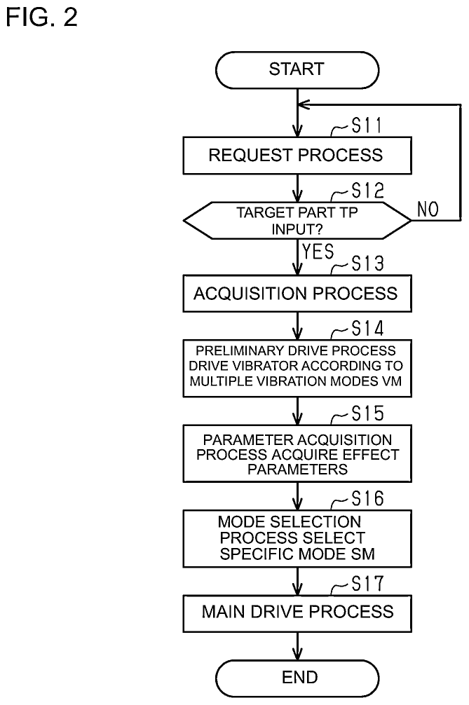

As illustrated in FIG. 2, after starting the control program P1, the CPU 41 first performs step S11. At step S11, the CPU 41 performs a request process. Specifically, in the request process, the CPU 41 outputs, to the input/output device 30, image data showing an input field for inputting a target part TP for which rehabilitation is requested and options for possible target parts TP. That is, the input/output device 30 displays, on the touch display, icons representing an input field for the target part TP and options for possible target parts TP. Then, the CPU 41 advances the process to step S12.

At step S12, the CPU 41 determines whether the target part TP for rehabilitation has been input by the user via the input/output device 30. Specifically, the CPU 41 determines which one of the options displayed in the request process has been selected. When one of the options has been selected, the CPU 41 determines that a user input has been received by the input/output device 30. On the other hand, when none of the options has been selected, the CPU 41 determines that no user input has been received by the input/output device 30.

When no user input has been received by the input/output device 30 (S12: NO), the CPU 41 returns the process to step S11. On the other hand, when a user input has been received by the input/output device 30 (S12: YES), the CPU 41 advances the process to step S13.

At step S13, the CPU 41 performs the acquisition process. In the acquisition process, the CPU 41 obtains information indicating the target part TP. Specifically, the CPU 41 obtains information indicating the target part TP selected via the input/output device 30 in the request process of step S11. Then, the CPU 41 advances the process to step S14.

At step S14, the CPU 41 starts the preliminary drive process. In the preliminary drive process, the CPU 41 drives the vibrator 20 in at least two of the multiple vibration modes VM in a predetermined order and for a predetermined preliminary period. In the present embodiment, the CPU 41 drives the vibrator 20 in all vibration modes VM stored in the storage device 44. Also, in the preliminary drive process, the CPU 41 causes the measurement device 70 to start the measurement of active states of the brain of the user. Here, the user can perform rehabilitation during the preliminary drive process while using the haptic sensation presentation device 10 as an assistive device. Then, the CPU 41 advances the process to step S15.

At step S15, the CPU 41 performs the parameter acquisition process. In the parameter acquisition process, the CPU 41 obtains, for each vibration mode VM, an effect parameter EP indicating the effectiveness of rehabilitation on the user achieved when the vibrator 20 is driven in the vibration mode VM in the preliminary drive process. In the present embodiment, the effect parameter EP indicates an activated region among multiple regions of the brain of the user that is activated to a predetermined level or higher. The predetermined level is determined in advance through a test or a simulation as a level indicating that the rehabilitation has a substantial effect. In particular, the effect parameter EP indicates the most activated region among multiple regions of the brain of the user. For example, a parameter X may correspond to a certain region of the brain, and a parameter Y may correspond to another region. In this way, the brain is sectioned into multiple regions, and a particular parameter is assigned to each of the regions. In determining the most activated region, the CPU 41 first identifies the largest value among the activation levels of the brain measured while the vibrator 20 is being driven in a first vibration mode VM. Next, the CPU 41 determines a region of the brain that showed the largest value as the most activated region. Next, similarly to the first vibration mode VM, the CPU 41 identifies the most activated region in a second vibration mode VM. The CPU 41 repeats this step for all vibration modes VM to calculate the most activated regions in the respective vibration modes VM. Then, the CPU 41 advances the process to step S16.

At step S16, the CPU 41 performs the mode selection process. In the mode selection process, the CPU 41 selects a specific mode SM, which is a specific vibration mode VM, from the multiple vibration modes VM based on the effect parameters EP obtained using the vibration modes VM in the parameter acquisition process.

Here, target parts TP, such as the right arm and the left leg on which rehabilitation is performed, correspond to regions of the brain that work when the target parts TP are moved. In the mode selection process, the CPU 41 selects, as the specific mode SM, a vibration mode VM with which the most activated region is located within a predetermined distance from a region that is among the multiple regions of the brain of the user and is activated when the target part TP is moved. The predetermined distance is determined in advance as a distance at which the target part TP is determined to be activated in a test or a simulation. Specifically, the predetermined distance is set at 10 mm. Particularly, in the present embodiment, the CPU 41 selects, as the specific mode SM, a vibration mode VM with which the most activated region becomes closest to a region that is among the multiple regions of the brain of the user and is activated when the target part TP is moved. Then, the CPU 41 advances the process to step S17.

At step S17, the CPU 41 performs the main drive process. In the main drive process, the CPU 41 drives the vibrator 20 in the specific mode SM selected in the mode selection process. In the main drive process, the CPU 41 drives the vibrator 20 in the specific mode SM for a predetermined main period. The main period is set longer than the preliminary period required for the preliminary drive process performed in the vibration mode VM selected as the specific mode SM. That is, the preliminary period is shorter than the main period. During the main drive process, the user performs rehabilitation on the target part TP by using the haptic sensation presentation device 10 as an assistive device. That is, the user performs exercises for rehabilitation while receiving the presentation of haptic sensations from the haptic sensation presentation device 10. Then, the CPU 41 ends the series of processes.

### Operation of Embodiment

According to the above embodiment, the user is presented with haptic sensations in the respective vibration modes VM in the preliminary drive process and thereby benefits from rehabilitation. Then, in the main drive process, the user is presented with a haptic sensation in the specific mode SM that is selected based on the effect parameters EP indicating the effectiveness of rehabilitation of the user in the preliminary drive process.

### Effects of Embodiment

(1) According to the above embodiment, the CPU 41 selects the specific mode SM in the mode selection process based on the effectiveness of rehabilitation of the user in the preliminary drive process. With this configuration, the CPU 41 can select an appropriate specific mode SM from among multiple vibration modes VM based on the effectiveness of rehabilitation of the user.
(2) According to the above embodiment, the CPU 41 performs the main drive process in the specific mode SM selected in the mode selection process. With this configuration, in the main drive process, the haptic sensation presentation device 10 can present an appropriate haptic sensation to the user based on the effectiveness of the rehabilitation of the user in the preliminary drive process without requiring operations by a person, such as a doctor or a physical therapist, having specialized knowledge. In other words, even in a place, such as the home of the user, where no healthcare professional is present, the user can receive the presentation of a haptic sensation in an appropriate vibration mode VM when performing rehabilitation.
(3) According to the above embodiment, the preliminary period is shorter than the main period. Therefore, the user does not have to wait for an excessively long time before performing the main drive process.
(4) When a haptic sensation is presented to the user, even if, for example, the arms and legs of the body of the user do not appear to move, a region in the primary motor cortex of the brain corresponding to the target part TP of the body may be activated. According to the above embodiment, the effect parameter EP indicates an activated region among multiple regions of the brain of the user that is activated to a predetermined level or higher. Therefore, the effect parameter EP can be obtained even when the body of the user does not appear to move.
(5) According to the above embodiment, the effect parameter EP indicates the most activated region among multiple regions of the brain of the user. Accordingly, effect parameters EP can be obtained such that each of the effect parameters EP corresponds to one vibration mode.

This in turn makes it possible to prevent the mode selection process from becoming excessively complex.
(6) According to the above embodiment, the specific mode SM selected in the mode selection process is a vibration mode VM with which the most activated region is located within a predetermined distance from a region that is among the multiple regions of the brain of the user and is activated when the target part TP is moved. This makes it possible to select, as the specific mode SM, a vibration mode VM that is likely to activate the target part TP of rehabilitation.
(7) According to the above embodiment, the specific mode SM selected in the mode selection process is a vibration mode VM with which the most activated region becomes closest to a region that is among the multiple regions of the brain of the user and is activated when the target part TP is moved. This makes it possible to select, as the specific mode SM, a vibration mode VM that is most likely to activate the target part TP of rehabilitation.

### Other Embodiments

The above embodiment may be modified and implemented as described below. The above embodiment and the variations described below may be combined as long as they do not technically conflict with each other.

The configuration of the vibrator 20 is not limited to that described in the above embodiment. For example, the vibrator 20 may be configured to use vibrations generated by a motor or may include a piezoelectric element.

The haptic sensation presentation device 10 may include multiple vibrators 20. In this case, the vibration mode VM may include information indicating which of the multiple vibrators 20 are caused to vibrate, the intensity of vibration, and the order in which the vibrators 20 are caused to vibrate. This makes it possible to increase the types of vibration modes VM.

In the above embodiment, the control device 40 is not limited to a device that includes a CPU and a ROM and executes software processes. For example, at least some of the software processes in the above embodiment may be implemented by a dedicated hardware circuit (such as ASIC) for hardware processing. That is, the control device 40 may have any of configurations (a) to (c) below. (a) The control device 40 includes a processing device that performs all of the above processes according to a program and a program storage device, such as a ROM, that stores the program. (b) The control device 40 includes a processing device that performs some of the above processes according to a program, a program storage device, and a dedicated hardware circuit that performs the remaining processes. (c) The control device 40 includes a dedicated hardware circuit that performs all of the above processes. Here, the control device 40 may include multiple software execution devices each of which includes a processing device and a program storage device, and/or multiple dedicated hardware circuits.

The request process may be configured to request the user to manually input characters without outputting options. The request process may be appropriately changed according to the configuration of the input/output device 30.

In the process of step S12, assuming that an input completion button is displayed on the input/output device 30, the completion of the input operation may be detected when the input completion button is pressed.

The effect parameter EP obtained in the parameter acquisition process is not limited to a parameter indicating the most activated region of the brain. The effect parameter EP may be any parameter indicating the effectiveness of rehabilitation on the user. For example, the effect parameter EP may indicate a most activated part of the body of the user other than the brain. For example, even rehabilitation for a target part TP may involve the movement of a part other than the target part TP. In such a case, it can be inferred that the effect of rehabilitation is high when the most activated region is close to the target part TP.

Also, for example, the effect parameter EP may indicate the activation level of a specific part of the body of the user. Examples of specific parts include regions of the brain in addition to arms and legs. There may be a case in which a specific part of the body of the user that needs to be focused on is known in advance based on the type of rehabilitation. In such a case, a vibration mode VM for a suitable haptic sensation can be selected as the specific mode SM by obtaining an effect parameter indicating the activation level of the specific part.

In the mode selection process, the CPU 41 just needs to select a specific mode SM from among the multiple vibration modes VM based on the effect parameters EP. Therefore, the CPU 41 just needs to select a specific mode SM according to the obtained effect parameters EP. For example, assume that each effect parameter EP indicates the activation level of a specific part of the body of the user as in the above-described variation. In this case, for example, in the mode selection process, the CPU 41 may select, as the specific mode SM, a vibration mode VM in which an effect parameter EP indicating the highest activation level is obtained.

In the mode selection process, the CPU 41 does not necessarily select a vibration mode based on the most activated region. For example, in the mode selection process, when the effect parameter EP indicates an activated region that is among multiple regions of the brain of the user and is activated to a predetermined level or higher, the CPU 41 may select the specific mode SM based on the activated region.

In the mode selection process, the CPU 41 does not necessarily select the specific mode SM with which the activated region becomes closest to a region that is activated when the target part TP is moved. In the mode selection process, the CPU 41 may select, as the specific mode SM, a vibration mode VM with which the activated region is located within the predetermined distance from a region that is among the multiple regions of the brain of the user and is activated when the target part TP is moved.

In the parameter acquisition process, there is a case in which multiple effect parameters EP are obtained in each vibration mode VM. In this case, in the mode selection process, the CPU 41 may select a specific mode SM with which one of the activated regions is located within the predetermined distance from a region that is among the multiple regions of the brain of the user and is activated when the target part TP is moved.

Assume that multiple effect parameters EP are obtained in each vibration mode VM in the parameter acquisition process. In this case, in the mode selection process, the CPU 41 may select, as the specific mode SM, a vibration mode VM with which the number of effect parameters EP obtained in the parameter acquisition process becomes greater than or equal to a predetermined value. With this configuration, the CPU 41 can select a mode that is highly likely to activate the target part TP by selecting, as the specific mode SM, a vibration mode VM that results in many activated regions.

Also, assume that multiple effect parameters EP are obtained in each vibration mode VM in the parameter acquisition process. In this case, in the mode selection process, the CPU 41 may select, as the specific mode SM, a vibration mode VM with which the number of effect parameters EP obtained in the parameter acquisition process becomes largest. With this configuration, the CPU 41 can select a mode that is most likely to activate the target part TP by selecting, as the specific mode SM, a vibration mode VM that results in many activated regions.
The main period of the main drive process may be changed as necessary. For example, the main period of the main drive process may be equal to the preliminary period of the preliminary drive process or may be shorter than the preliminary period.
Performing the main drive process after the preliminary drive process is optional. For example, the type of the specific mode SM determined through the preliminary drive process may be displayed on the input/output device 30. In this case, the user may specify the specific mode SM on the haptic sensation presentation device 10.

The haptic sensation presentation device 10 may store the specific mode SM determined in the mode selection process. When the specific mode SM is stored, it is desirable to be able to select whether to perform the main drive process without going through the preliminary drive process or to perform both of the preliminary drive process and the main drive process.

The measurement device 70 is not limited to a brain measuring instrument using near-infrared spectroscopy. For example, the measurement device 70 may be a measuring instrument using functional magnetic resonance imaging (functional MRI). As another example, the measurement device 70 may be a measuring instrument using electroencephalography (EEG).

The measurement device 70 is not necessarily a device for measuring the active state of the brain of the user. For example, the measurement device 70 may be configured to measure the active state of a specific part of the body of the user other than the brain. For example, when a target part of rehabilitation of the user is the right leg, the measurement device 70 may be configured to measure the active state of the right leg.

Technical ideas conceivable from the above embodiment and variations are additionally described below.

### <Appendix 1>

A control device for a vibrator of a haptic sensation presentation device includes a storage device and an execution device. The storage device stores multiple vibration modes of the vibrator that are used to present haptic sensations output by the haptic sensation presentation device for rehabilitation. The execution device performs a preliminary drive process of driving the vibrator in the multiple vibration modes to present the haptic sensations in at least two vibration modes among the multiple vibration modes, a parameter acquisition process of obtaining effect parameters for the respective vibration modes, the effect parameters indicating effectiveness of the rehabilitation on a user achieved when the vibrator is driven in the respective vibration modes in the preliminary drive process, and a mode selection process of selecting a specific mode, which is a specific vibration mode, from among the multiple vibration modes based on the effect parameters obtained in the respective vibration modes in the parameter acquisition process.

### <Appendix 2>

The control device for the vibrator of the haptic sensation presentation device according to appendix 1. The execution device further performs a main drive process of driving the vibrator in the specific mode selected in the mode selection process.

### <Appendix 3>

The control device for the vibrator of the haptic sensation presentation device according to appendix 2. A period required to perform the preliminary drive process in the vibration mode selected as the specific mode is shorter than a period required to perform the main drive process.

### <Appendix 4>

The control device for the vibrator of the haptic sensation presentation device according to any one of appendices 1 to 3. Each of the effect parameters indicates an activated region that is among multiple regions of a brain of the user and is activated to a predetermined level or higher.

### <Appendix 5>

The control device for the vibrator of the haptic sensation presentation device according to appendix 4. Each of the effect parameters indicates a most activated region among activated regions.

### <Appendix 6>

The control device for the vibrator of the haptic sensation presentation device according to appendix 4 or 5. The execution device further performs an acquisition process of obtaining information indicating a target part on which the rehabilitation is performed. In the mode selection process, the execution device selects the specific mode with which the activated region is located within a predetermined distance from a region that is among the multiple regions of the brain of the user and is activated when the target part is moved.

### <Appendix 7>

The control device for the vibrator of the haptic sensation presentation device according to appendix 6. In the mode selection process, the execution device selects a vibration mode with which the activated region becomes closest to the region that is among the multiple regions of the brain of the user and is activated when the target part is moved.

### <Appendix 8>

The control device for the vibrator of the haptic sensation presentation device according to appendix 4. The execution device further performs an acquisition process of obtaining information indicating a target part on which the rehabilitation is performed. When multiple effect parameters are obtained in each of the vibration modes in the parameter acquisition process, the execution device selects, in the mode selection process, the specific mode with which one of multiple activated regions is located within a predetermined distance from a region that is among the multiple regions of the brain of the user and is activated when the target part is moved.

### <Appendix 9>

The control device for the vibrator of the haptic sensation presentation device according to appendix 4. When multiple effect parameters are obtained in each of the vibration modes in the parameter acquisition process, the execution device selects, in the mode selection process, the specific mode with which the number of the effect parameters obtained in the parameter acquisition process becomes greater than or equal to a predetermined vale.

### <Appendix 10>

The control device for the vibrator of the haptic sensation presentation device according to appendix 4 or 9. When multiple effect parameters are obtained in each of the vibration modes in the parameter acquisition process, the execution device selects, in the mode selection process, the specific mode with which the number of the effect parameters obtained in the parameter acquisition process becomes largest.

### <Appendix 11>

The control device for the vibrator of the haptic sensation presentation device according to any one of appendices 1 to 3. Each of the effect parameters indicates an activation level of a specific part of a body of the user.

### <Appendix 12>

The control device for the vibrator of the haptic sensation presentation device according to appendix 11. In the mode selection process, the execution device selects one of the vibration modes with which an effect parameter indicating a highest activation level is obtained.

### <Appendix 13>

A haptic sensation presentation device includes a vibrator and a control device that includes a storage device and an execution device and controls the vibrator. The storage device stores multiple vibration modes of the vibrator that are used to present haptic sensations output for rehabilitation. The execution device performs a preliminary drive process of driving the vibrator in the multiple vibration modes to present the haptic sensations in at least two vibration modes among the multiple vibration modes, a parameter acquisition process of obtaining effect parameters for the respective vibration modes, the effect parameters indicating effectiveness of the rehabilitation on the user achieved when the vibrator is driven in the respective vibration modes in the preliminary drive process, and a mode selection process of selecting a specific mode, which is a specific vibration mode, from among the multiple vibration modes based on the effect parameters obtained in the respective vibration modes in the parameter acquisition process.

### REFERENCE SIGNS LIST

10 haptic sensation presentation device
20 vibrator
30 input/output device
40 control device
41 CPU
42 peripheral circuit
43 ROM
44 storage device
45 bus
70 measurement device
100 haptic sense presentation system
EP effect parameter
P1 control program
SM specific mode
TP target part
VM vibration mode
VMD vibration mode data

1. A control device for a vibrator of a haptic sensation presentation device comprising:
a storage device; and
a processor,
wherein the storage device is configured to store a plurality of vibration modes of the vibrator that present haptic sensations for rehabilitation; and
wherein the processor is configured to perform:
a preliminary drive process of driving the vibrator in the plurality of vibration modes to output the haptic sensations in at least two vibration modes among the plurality of vibration modes,
a parameter acquisition process of obtaining a plurality of effect parameters for the respective vibration modes, wherein the plurality of effect parameters indicate an effectiveness of the rehabilitation of a user achieved when the vibrator is driven in the respective vibration modes in the preliminary drive process, and
a mode selection process of selecting a specific mode, which is a specific vibration mode from among the plurality of vibration modes based on the plurality of effect parameters obtained in the parameter acquisition process.

2. The control device for the vibrator of the haptic sensation presentation device according to claim 1, wherein the processor is further configured to perform a main drive process of driving the vibrator in the specific mode selected in the mode selection process.

3. The control device for the vibrator of the haptic sensation presentation device according to claim 2, wherein a period for performing the preliminary drive process in the vibration mode selected as the specific mode is shorter than a period for performing the main drive process.

4. The control device for the vibrator of the haptic sensation presentation device according to claim 1, wherein each of the effect parameters indicates an activated region that is among a plurality of regions of a brain of the user and is activated to a predetermined level or higher.

5. The control device for the vibrator of the haptic sensation presentation device according to claim 4, wherein each of the effect parameters indicates a most activated region among activated regions of the brain.

6. The control device for the vibrator of the haptic sensation presentation device according to claim 4,
wherein the processor is further configured to perform an acquisition process of obtaining information indicating a target part of the body of the user; and
wherein in the mode selection process, the processor is configured to select the specific mode with which the activated region is located within a predetermined distance from a region that is among the plurality of regions of the brain of the user and is activated when the target part is moved.

7. The control device for the vibrator of the haptic sensation presentation device according to claim 6, wherein in the mode selection process, the processor is configured to select a vibration mode with which the activated region becomes closest to the region and is activated when the target part is moved.

8. The control device for the vibrator of the haptic sensation presentation device according to claim 4,
wherein the processor is further configured to perform an acquisition process of obtaining information indicating a target part of the body of the user; and
wherein when the plurality of effect parameters are obtained in each of the vibration modes in the parameter acquisition process, the processor is configured to select, in the mode selection process, the specific mode with which one of the plurality of activated regions is located within a predetermined distance from a region that is among the plurality of regions of the brain of the user and is activated when the target part is moved.

9. The control device for the vibrator of the haptic sensation presentation device according to claim 4, wherein when multiple effect parameters are obtained in each of the vibration modes in the parameter acquisition process, the processor is configured to select, in the mode selection process, the specific mode with which the plurality of effect parameters obtained in the parameter acquisition process becomes greater than or equal to a predetermined vale.

10. The control device for the vibrator of the haptic sensation presentation device according to claim 4, wherein when the plurality of effect parameters are obtained in each of the vibration modes in the parameter acquisition process, the processor is configured to select, in the mode selection process, the specific mode with which the number of the effect parameters obtained in the parameter acquisition process becomes largest.

11. The control device for the vibrator of the haptic sensation presentation device according to claim 1, wherein each of the effect parameters indicates an activation level of a specific part of the body of the user.

12. The control device for the vibrator of the haptic sensation presentation device according to claim 11, wherein in the mode selection process, the processor is configured to select one of the vibration modes with which an effect parameter indicating a highest activation level is obtained.

13. A haptic sensation presentation device comprising:
a vibrator; and
a control device comprising a storage device and a processor, and configured to control the vibrator,
wherein the storage device is configured to store a plurality of vibration modes of the vibrator that present haptic sensations output for rehabilitation; and
wherein the processor is configured to perform:
a preliminary drive process of driving the vibrator in the plurality of vibration modes to output the haptic sensations in at least two vibration modes among the plurality of vibration modes,
a parameter acquisition process of obtaining a plurality of effect parameters for the respective vibration modes, wherein the plurality of effect parameters indicate the effectiveness of the rehabilitation on the user achieved when the vibrator is driven in the respective vibration modes in the preliminary drive process, and
a mode selection process of selecting a specific mode, which is a specific vibration mode, from among the plurality of vibration modes based on the plurality of effect parameters obtained in the parameter acquisition process.

14. The control device for the vibrator of the haptic device according to claim 2, wherein each of the effect parameters indicates an activated region that is among a plurality of regions of a brain of the user and is activated to a predetermined level or higher.

15. The control device for the vibrator of the haptic device according to claim 3, wherein each of the effect parameters indicates an activated region that is among a plurality of regions of a brain of the user and is activated to a predetermined level or higher.

16. The control device for the vibrator of the haptic device according to claim 5,
wherein the processor is further configured to perform an acquisition process of obtaining information indicating a target part of the body of the user; and
wherein in the mode selection process, the processor is configured to select the specific mode with which the activated region is located within a predetermined distance from a region that is among the plurality of regions of the brain of the user and is activated when the target part is moved.
